(19) United States
(12) Plant Patent Application Publication
(10) Pub. No.: US 2013/0269073 P1
(43) Pub. Date: Oct. 10, 2013

(54) Coastal redwood tree named 'Semper 95'

(50) ;

(71) Applicant: John Clark, Summerville, SC (US)

(72) Inventor: John Clark, Summerville, SC (US)

(21) Appl. No.: 13/506,266

(22) Filed: Apr. 9, 2012

(51) Int. Cl. A01H 7/00

(52) U.S. Cl. PLT/213

(57) ABSTRACT

A new and distinctive variety of a coastal redwood tree designated as “Semper 95.” Semper 95 is distinguished by high tolerance to both low and high temperature, excellent stem straightness, and very fast growth.

### CROSS REFERENCE TO RELATED APPLICATIONS

Not Applicable.

### STATEMENT REGARDING FEDERALLY SPONSERED RESEARCH AND DEVELOPMENT

Not Applicable

### LATIN NAME

Sequoia sempervirens

### VARIETY DENOMINATION

“Semper 95”

### BACKGROUND OF THE INVENTION

A new variety of coastal redwood tree (Sequoia sempervirens), has been discovered. This variety has been designated as “Semper 95.” The market class of this plant is PLT/213.

This new variety was discovered during field trials of open pollinated seedlings conducted in Summerville, S.C. between 1994 and 1999.

The seedlings were obtained from the Glass Mountain Forest Tree Nursery (P.O. Box 440, St. Helena, Calif. 94574). Approximately 100 seedlings were established each year during the spring in one gallon containers. After one year cuttings were taken from surviving plants and established in one gallon containers.

Over the five year screening period six cultivars survived the initial environmental field screening and subsequent micropropagation process. These cultivars were established as rooted cuttings. Of the six cultivars, five demonstrated increased tolerance to heat, but only a moderate increase in tolerance to cold resulting in significant die back and cold damage when temperatures fell below 32 F. The damage inflicted by the cold impacted tree form by induced forking which is undesirable in a commercial saw-timber tree. Overall growth rate of this subset of survivors was unremarkable.

The sixth surviving cultivar, designated “Semper 95” possessed impressive heat/cold tolerance to the degree that cold damage has never been observed on the parent plant nor on any rooted cuttings derived from this tree. Additionally “Semper 95” and rooted cutting derived clones have demonstrated an excellent growth rate and form making this tree a candidate for commercial forestry.

First rooted cuttings of this variety were produced in 1997. Ramets were planted at several field test sites in South Carolina.

### BRIEF SUMMARY

This new and distinct cultivar of coastal redwood (Sequoia Sempervirens) is distinctly characterized by its significantly increased tolerance to high and low temperature, while maintaining a very high growth rate. Additionally this variety possesses excellent stem straightness. These characteristics make this variety suitable and desirable for commercial forestry in an expanded range of climates and most notably in the southeast portion of the United States.

The Sequoia sempervirens plants of this variety were asexually propagated by creating rooted cuttings of rapidly growing shoots. Other methods of propagation may also be employed such as micropropagation of auxenic explants in tissue culture or through somatic embryogenesis.

Rooted cuttings of this variety have reproduced the traits of the primary specimen over several growing seasons.

### BRIEF DESCRIPTION OF THE DRAWINGS

The drawings are color photographs showing the parental ramet of the new variety of coastal redwood.

FIG. 1 is a photograph showing a 13 year old specimen planted in Summerville, S.C. The picture shows the excellent stem straightness, the basic tree form and coloring. For scale, the staff with the bright green tip in front of the trunk is 66 inches long.

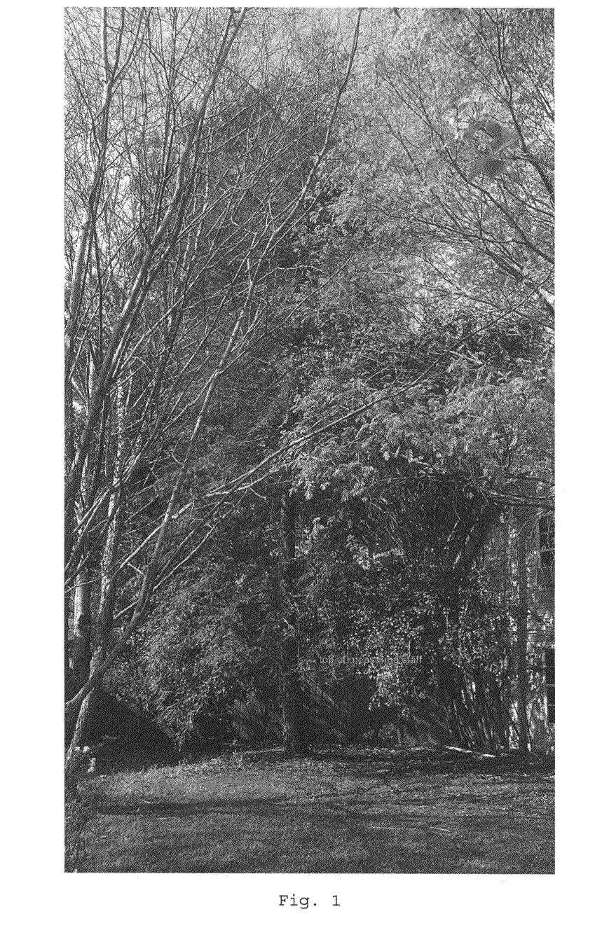

FIG. 2 is a photograph showing needle morphology, size and color.

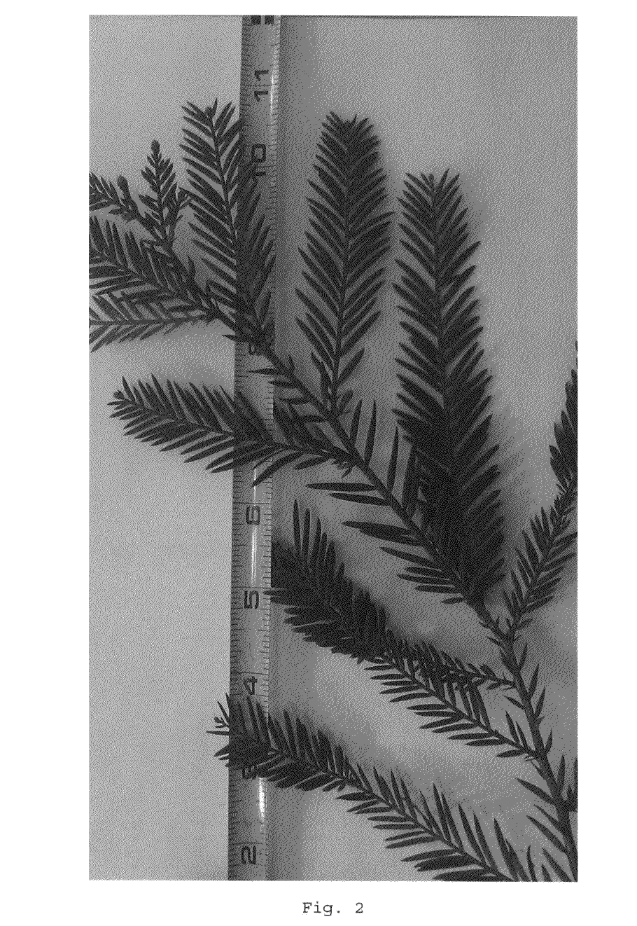

FIG. 3 is a photograph showing the trunk and tree form from a different angle.

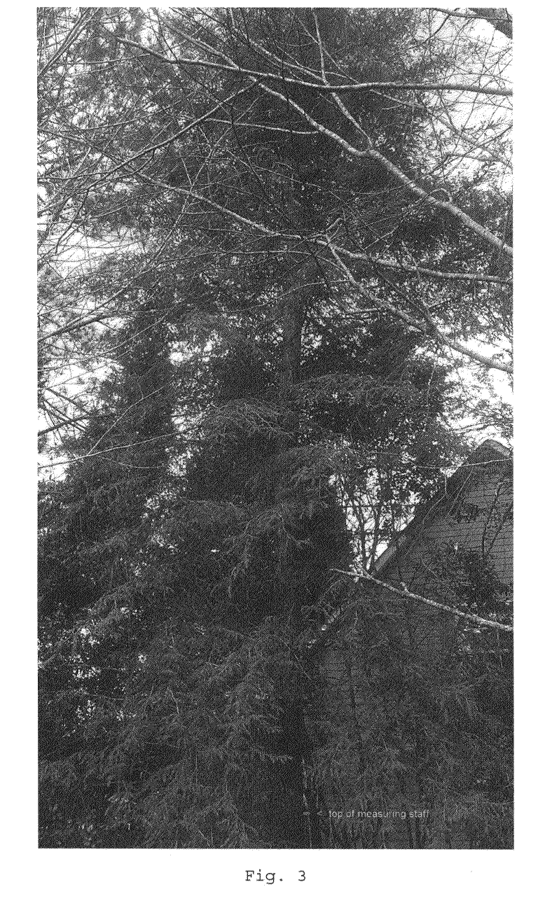

FIG. 4 is a photo taken from the base of the tree showing the straightness characteristic of the trunk.

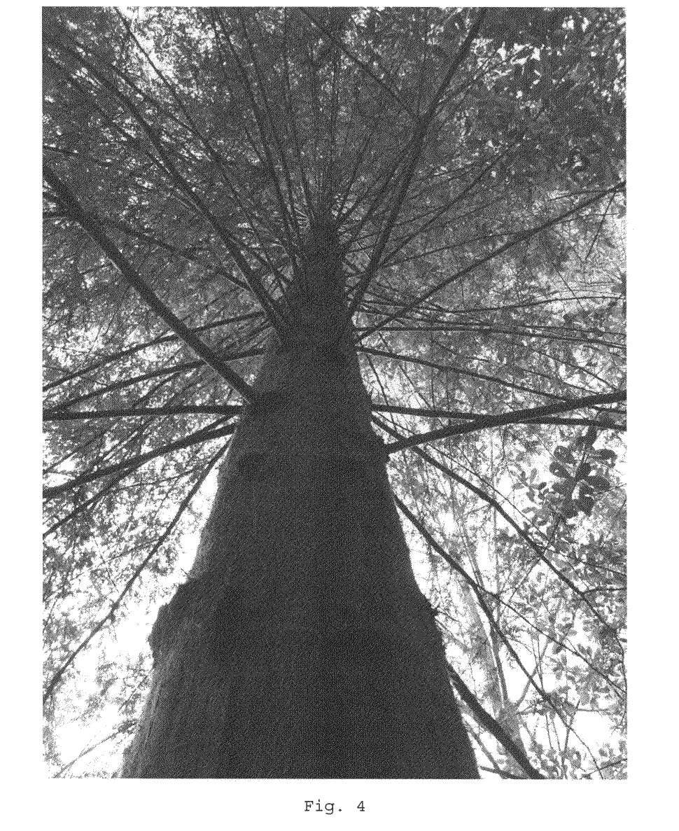

### DETAILED BOTANICAL DESCRIPTION

The botanical details of this new and distinct variety of coastal redwood tree are as follows.
Leaf: Evergreen needles, approximately ¾ inch long, 1/16 inch wide, arranged as a fan along a leaf between 2 and 5.5 inches long. Needles are light green then turn a darker green as they age.
Flower: Monoecious; males are small cylindrical, light green then yellow at maturity, forming individually at branch tips; females are small spherical green initially then turning brown at maturity.
Seed: Brown-red in color, ovoid in shape, approximately 1.5-2 mm in diameter, maturing in early fall.
Twig: Orange-brown in color, very flexible; buds are narrowly ovoid, light green in color.
Bark: Initially red on new growth becoming orange-brown and scaly. On the trunk between the scales the bark becomes soft and fibrous. The bark of older trees becomes darker and thicker.
Form: A medium aged tree 15-17 years old can reach 50 feet; an older tree 30+ years tree can reach well over 300 feet tall. This variety quickly develops a straight trunk with minimal buttressing and a conical apical crown.
Temperature tolerance: Semper 95 as a juvenile rooted cutting and as an established tree exhibits cold tolerance through temperatures at least as low as 19 F. Established cuttings and trees tolerate temperatures as high as 105 F. Based on its performance in test plots it is anticipated that this variety possesses a cold tolerance range exceeding that described here. Unfortunately the methods employed to discover the variety were not designed to test the limits of the specimen's cold tolerance.
Average height: 41 ft after 13 field growing seasons. Average diameter at breast height: 13.3 inches after 13 field growing seasons. Propagation: propagated by rooted cutting.
Use: Private ornamental and commercial-industrial tree farms.

Compared to unimproved coastal redwood trees, ‘Semper 95’ is characterized by very high growth rate coupled with significantly increased range of temperature tolerance and excellent stem straightness.

The new variety of coastal redwood tree possesses the defined characteristics described above as a result of the growing conditions prevailing at the primary test site, some variation should be expected incident to changes in growing conditions, irrigation, fertilization, pruning, pest control, climatic variations and the like.

1. A new and distinct variety of coastal redwood named Semper 95 as described and illustrated.
